(19) United States
(12) Patent Application Publication
(10) Pub. No.: US 2008/0171988 A1
(43) Pub. Date: Jul. 17, 2008

(54) DOUBLE-CONE SPHINCTER INTRODUCER ASSEMBLY AND INTEGRATED VALVE ASSEMBLY

(75) Inventor: Ernesto E. Blanco, Belmont, MA (US)

Correspondence Address: OBLON, SPIVAK, MCCLELLAND MAIER & NEUSTADT, P.C., 1940 DUKE STREET, ALEXANDRIA, VA, 22314, US

(73) Assignee: Erblan Surgical, Inc., Belmont, MA (US)

(21) Appl. No.: 11/931,846

(22) Filed: Oct. 31, 2007

Related U.S. Application Data
(60) Provisional application No. 60/885,322, filed on Jan. 17, 2007.

(51) Int. Cl. A61B 17/00

(52) U.S. Cl. 604/167.03

(57) ABSTRACT

A seal assembly includes a first seal, a second seal, and a positioning element. The first seal includes at least one longitudinal slot therein. The second seal includes at least one longitudinal slot therein and is positioned coaxially with the first seal. The positioning element is positioned coaxially with the first seal. The at least one longitudinal slot of the first seal is offset from the at least one longitudinal slot of the second seal in a radial direction from a longitudinal axis of the seal assembly. The seal assembly can be integrated with a closing valve. The seal assembly can also be integrated with a capsule.

### CROSS-REFERENCE TO RELATED APPLICATIONS

This application claims the benefit of the earlier filing date of U.S. Provisional Application No. 60/885,322, filed Jan. 17, 2007, the entirety of which is incorporated herein by reference.

### BACKGROUND OF THE INVENTION

1. Field of the Invention

The present invention relates to a double-cone sphincter introducer assembly and an integrated valve assembly utilized, for example, in a trocar and provides distinct advantages with respect to the ability to effectively seal a cannula of the trocar while having sufficient flexibility and resilience to permit the insertion of an obturator of substantially any size less than the diameter of the cannula. This assembly also permits surgical instruments, such as a clip applicator and the clips utilized in surgery, to be inserted therethrough and retracted or withdrawn therefrom upon conclusion of surgery without loss of insufflation pressure within the patient or the passage of fluids from the distal end to the proximate end of the trocar during surgery. The seal of the present invention constitutes an improvement over the seal shown in Applicant's earlier issued patent, U.S. Pat. No. 6,497,687, and can also be utilized in the trocar appearing in U.S. Pat. No. 6,719,746 to Applicant, the entirety of each of which is incorporated by reference herein.

2. Description of Related Art

Seals known in the art are characterized by the drawback of being torn or cut upon removing surgical instruments, such as the clips of clip applicators, from a patient. The result is a loss of pressure in an insufflated portion of a patient and the ejection of fluids past the seal. Thus, the need for an effective seal to overcome this problem has become necessary.

### SUMMARY OF THE INVENTION

An object of the present invention is to provide an improved seal and valve assembly for a trocar or other instrument so as to permit the passage of and withdrawal of an obturator, a clip applicator, and other surgical instruments therethrough while maintaining an effective seal for preventing the escape of gas and fluids within the portion of a patient located beneath the peritoneum of the body cavity. It is envisioned, however, that the present invention can be utilized for other purposes where an effective seal is needed while permitting the passage through the seal and withdrawal of an object back through the seal in a controlled and effective manner and to maintain the sealing capacity of the seal.

In view of the foregoing, Applicant has invented the double cone sphincter introducer assembly and integrated valve assembly described below and as illustrated in the attached FIGS. 1-16. In this regard, it is to be noted that the double cone sphincter introducer assembly illustrated in FIGS. 6-10, or any of the exemplary embodiments thereof, can be incorporated in the rear or proximal end of the seal assembly illustrated in FIGS. 4 and 5, or any of the exemplary embodiments thereof, as explained in detail hereinbelow.

### DESCRIPTION OF THE DRAWINGS

FIG. 1A illustrates an exemplary closing valve;

FIG. 1B illustrates a cross sectional view of the closing valve taken at section A-A of FIG. 1A;

FIG. 1C illustrates the exemplary closing valve of FIG. 1A integrated with a capsule;

FIG. 2 illustrates an exemplary integrated closing valve and seal assembly;

FIG. 3 illustrates an exemplary capsule for a closing valve;

FIG. 4 illustrates an exemplary integrated closing valve and seal assembly positioned within an exemplary capsule;

FIG. 5 illustrates an exemplary integrated closing valve and seal assembly integrated with an exemplary capsule;

FIG. 6 illustrates a sectional view taken along an axis of the exemplary integrated closing valve and seal assembly shown in FIG. 2;

FIG. 7 illustrates a sectional view taken along an axis of an exemplary integrated closing valve and seal assembly with cones integrated thereto;

FIG. 8 illustrates a first view of a surgical tool entering an exemplary seal assembly;

FIG. 9 illustrates a second view of a surgical tool entering an exemplary seal assembly;

FIG. 10A illustrates an isolated perspective view of an exemplary seal assembly;

FIG. 10B illustrates a cross sectional view of the isolated seal assembly taken at section A-A of FIG. 10A;

FIG. 11 illustrates an exemplary mold for forming an integrated closing valve and a seal assembly;

FIG. 12 illustrates an integrated closing valve and a seal assembly formed in the mold illustrated in FIG. 11;

FIG. 13 illustrates another exemplary closing valve;

FIG. 14A illustrates another exemplary seal assembly;

FIG. 14B illustrates a sectional view taken along an axis of the exemplary seal assembly shown in FIG. 14A;

FIG. 15 is another illustration of the exemplary seal assembly shown in FIG. 14A and FIG. 14B;

FIG. 16A illustrates another exemplary seal assembly and exemplary capsule integrated with a cannula;

FIG. 16B is another illustration of the capsule shown in FIG. 16A;

FIG. 16C illustrates another exemplary embodiment of a seal assembly and capsule integrated with a cannula;

FIG. 16D illustrates another exemplary embodiment of a seal assembly and capsule integrated with a cannula;

FIG. 16E illustrates another exemplary embodiment of a seal assembly and capsule integrated with a cannula; and

FIG. 16F illustrates an exemplary embodiment of an integrated seal assembly integrated with an exemplary capsule.

### DESCRIPTION OF THE PREFERRED EMBODIMENTS

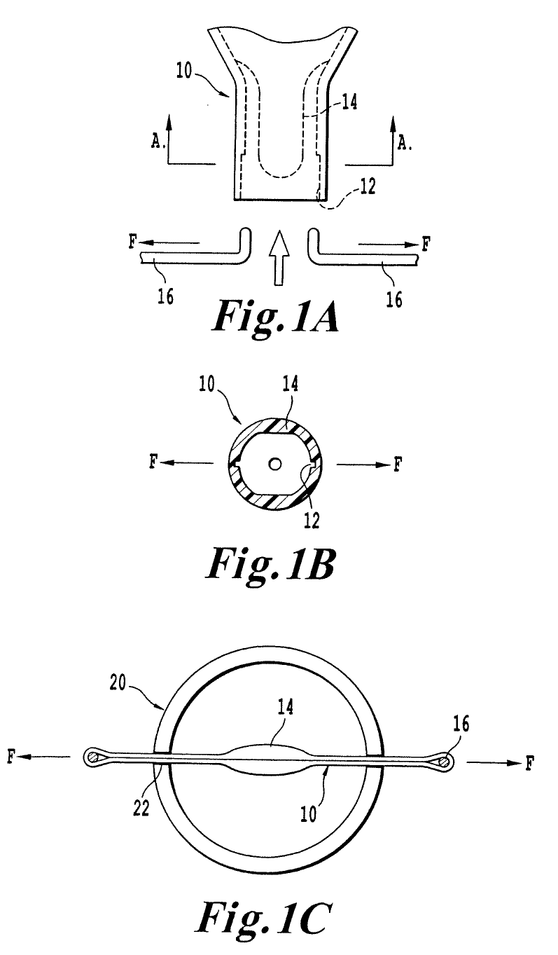

With reference to the figures, FIG. 1A illustrates an exemplary closing valve 10. FIG. 1B is a cross sectional view of the closing valve 10 shown in FIG. 1A taken at section A-A. The end of the closing valve 10 can be molded with two short semicircular inside grooves 12 located at an inner edge of the closing valve 10 to facilitate an accurate folding thereof with equal stresses at a top and bottom portion 14 thereof. In addition, the top and bottom portion 14 can be made thicker that the rest of the closing valve 10 to prevent snagging by sharp tools while at the same time improving the closure effect.

The structure illustrated in FIG. 1A shows the closing valve 10 with inside semicircular cylinder grooves 12 and thicker walls in between. The structure illustrated in FIG. 1A also shows a technique for stretching the edges of the closing valve 10 by using two hooks 16 inserted into the inside grooves 12 and pulled outwardly by stretching forces F after the hooks 16 are inserted into the inside grooves 12 as shown by the center arrow.

The structure illustrated in FIG. 1C is an end view of the closing valve 10 integrated with a capsule 20 and showing the action of the stretching forces F which effectively thin-down the closing valve 10 edges to facilitate their insertion into the slots 22 at the respective ends of the capsule 20. Thereafter, the outside of the inserted closing valve 10 protruding from the capsule slots 22 can be bonded and trimmed; the bonding can be performed at the outside portion thereof. A material suitable for such an integration process, such as silicone, can be used for the closing valve 10.

The result of the assembly of the closing valve 10 and the capsule 20 is a tough, resilient, and effective valve closure independent of gas pressure for its closing effect. A novel and reliable closing valve 10 thus is formed which overcomes the drawbacks of conventional seal assembles.

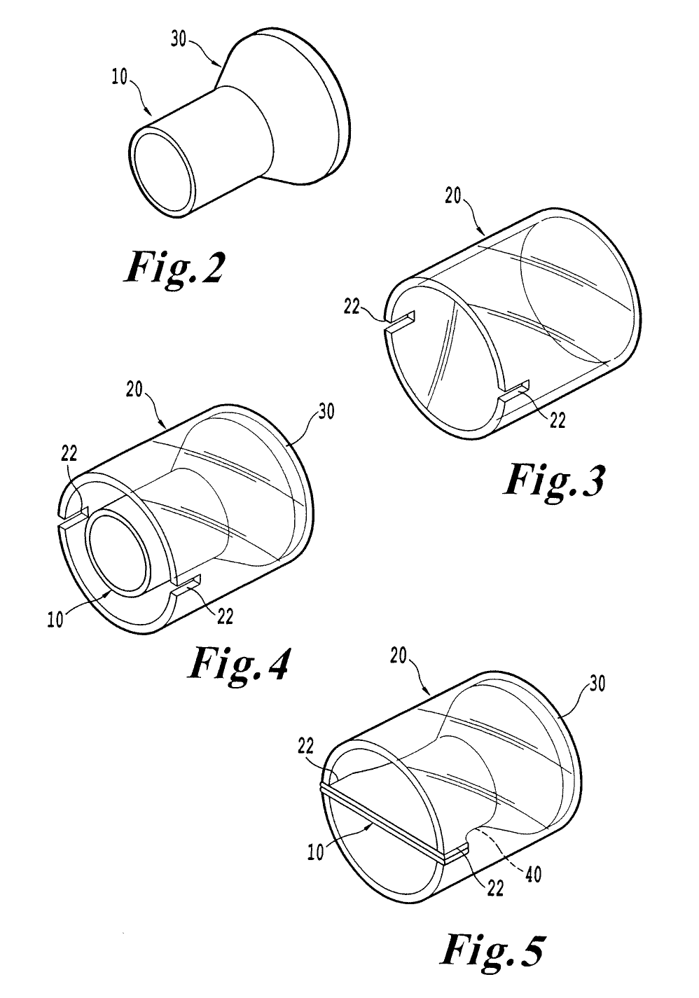

FIG. 2 illustrates an exemplary integrated closing valve 10 and seal assembly 30. The seal assembly 30 will be more fully described below. FIG. 3 illustrates an exemplary capsule 20 in which an integrated closing valve 10 and seal assembly 30 can be integrated. FIG. 4 illustrates an exemplary integrated closing valve 10 and seal assembly 30 positioned within an exemplary capsule 20. The plastic capsule 20 is insertable into the cannula of a trocar. As can be seen in FIG. 4, the closing valve 10 is not integrated with the capsule 20.

FIG. 5 illustrates an exemplary integrated universal closure showing the closing valve 10 in a closed position such that the area where the closing valve 10 integrates with the seal assembly 30 forms an hourglass seal neck 40. The edges of the closing valve 10 shown in FIG. 5 are lightly stretched, inserted into the slots 22 of the capsule 20, and are then bonded and trimmed by a suitable method known to a person of ordinary skill in the art so as to maintain the position of the closing valve 10 within the plastic capsule 20. While a plastic capsule 20 has been illustrated, it is of course understood that a metal capsule or other suitable material can be utilized that provides the proper support and positioning of the seal assembly 30.

Additionally, in an alternative embodiment, the capsule can be fixed to the seal assembly and does not need to be fixed to the closing valve. For example, the outer periphery of the seal assembly can be adhered or fixed to the inner surface of the capsule as illustrated in FIGS. 4 and 5. Further, alternative means, such as clamps or heat fusing could be used to keep the ends of the closing valve together.

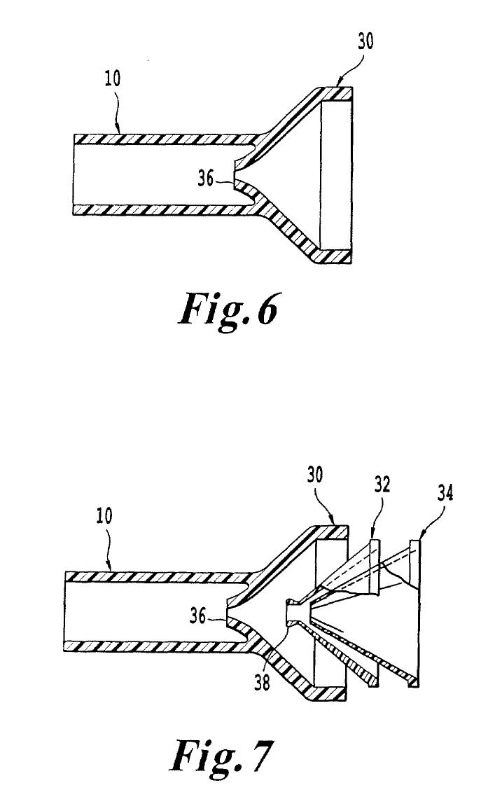

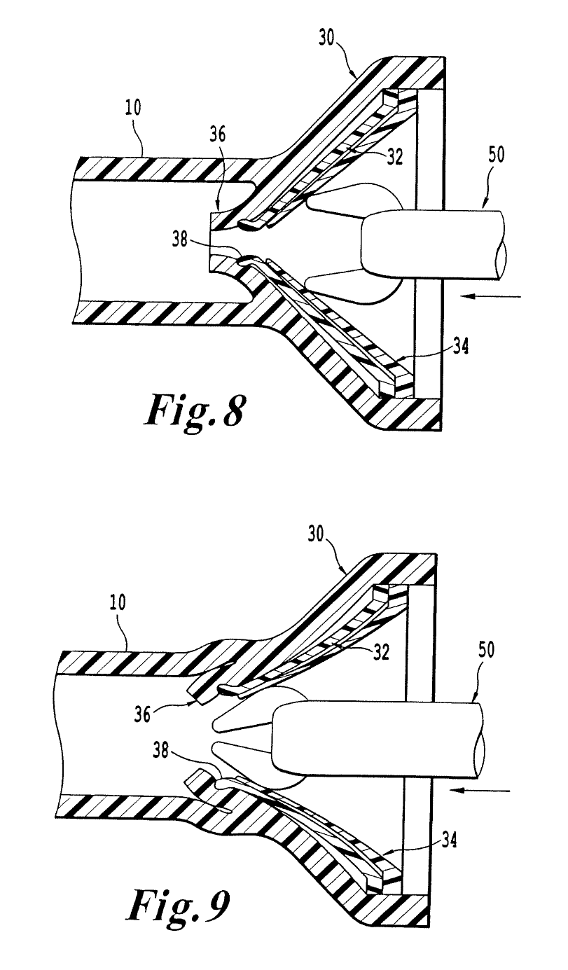

As can thus be appreciated, FIGS. 1-5 illustrate various embodiments of the structure of the integrated valve assembly formed by the closing valve 10 and the seal assembly 30, into which a double-cone sphincter introducer assembly of the type illustrated in FIGS. 6-10 is positioned. As can be appreciated from a review of FIGS. 6-10, FIG. 6 illustrates a cross-sectional view taken along an axis of the exemplary integrated closing valve 10 and seal assembly 30 shown in FIG. 2, and FIG. 7 illustrates a cross-sectional view taken along an axis of the exemplary integrated closing valve 10 and seal assembly 30 with at least the first and second seals 32, 34 integrated thereto. As can be seen, both the first and second seals 32, 34 have a substantially conical shape. Additional cones (not shown) could also be integrated thereto. FIGS. 8 and 9 illustrate the manner in which a large surgical tool 50 is able to enter an exemplary seal assembly 30 such that at least two seals 32, 34 are pushed together and against a closing portion 36 of the seal assembly 30. The closing portion 36 of the seal assembly 30 form a positioning element for the first and second seals 32, 34. The closing portion 36 of the seal assembly 30 and the first and second seals 32, 34 form a sphincter assembly such that additional pushing serves to further open the closing portion 36 of the seal assembly 30 and perhaps partly close the tool 50, as can be seen in FIG. 9, while sparing the seal assembly 30 from damage. As can be seen, the closing portion 36 and the at least two seals 32, 34 can all be coaxial and the seals 32, 34 can have a substantially conical shape.

As shown in FIG. 8, the second seal 34 extends radially a shorter distance than the first seal 32 so as to remain out of the path of outgoing tools 50 that could snag on the edges of the second seal 34. The inner rim 38 of the first seal 32 has a substantially cylindrical shape to engage the closing portion 36 of the seal assembly 30 and ensure clear passage of tools 50 in any direction while maintaining the effective action of the closing portion 36 of the seal assembly 30 contacting the tools 50. In the exemplary system shown herein, the closing portion 36 of the seal assembly 30 is not opened by any contact with a surgical tool 50. The opening is accomplished by the action of the sphincter assembly including the first and second seals 32, 34. For example, a surgical tool 50 entering the seal assembly 30 first contacts the second seal 34 which will contact the first seal 32. The first seal 32, and specifically the inner rim 38 of the first seal 32, contacts the closing portion 36, causing the closing portion 36 to open and allow the tool 50 to pass therethrough. Only the second seal 34, the inner rim 38 of the first seal 32, and the closing portion 36 of the seal assembly 30 ever contact the incoming surgical tools 50. Thus, tools 50 can move freely in and out of the seal assembly 30 without damaging the seals 32, 34 or the closing portion 36.

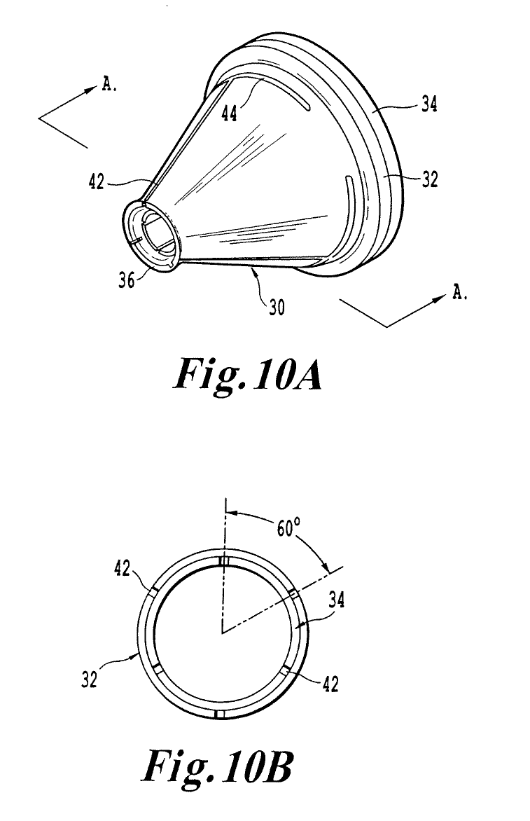

To facilitate opening of the seal assembly 30, the seals 32, 34 can be segmented with three slots 42 (see FIGS. 10A and 10B) lengthwise at an angle of approximately 120° apart, for example. At the base end of the seals 32, 34, the lengthwise slots 42 terminate against a circumferential slot 44 to permit their outward radial flexure when forced by an entering tool 50. The lengthwise slots 42 of each of the seals 32, 34 allow opening of the sphincter assembly and can be assembled directly between the lengthwise slots 42 of the mating seal 32, 34 to prevent surgical tools 50 from slipping out during radial expansion. The lengthwise slots 42 of the seals 32, 34 can be offset from one another by approximately 60°, for example. Therefore, a tool 50 of any shape can be introduced into the closure of the seal assembly 30 at any angle. Thus, the action of the sphincter assembly is substantially radial, resulting in novel opening kinematics compared to those of conventional systems.

The seals 32, 34 shown in the figures are relatively thin, but can be made of tough plastic and have an annular base rim. The seals 32, 34 can be made of any suitable low friction plastic, such as TEFLON™.

FIGS. 10A and 10B serve to provide an isolated visual illustration of an exemplary seal assembly 30 which utilizes the two seals 32, 34, one placed inside the other so as to form the double-cone sphincter introducer assembly. The two seals 32, 34 can be attached to one another, for example via bonding or gluing, or the two seals 32, 34 can be removably force fitted to one another. The double-cone sphincter introducer assembly can then be secured and sealed by an outer ring, or other suitable securing device or method, to the integrated closing valve 10 and seal assembly 30 as shown in FIG. 2, for example.

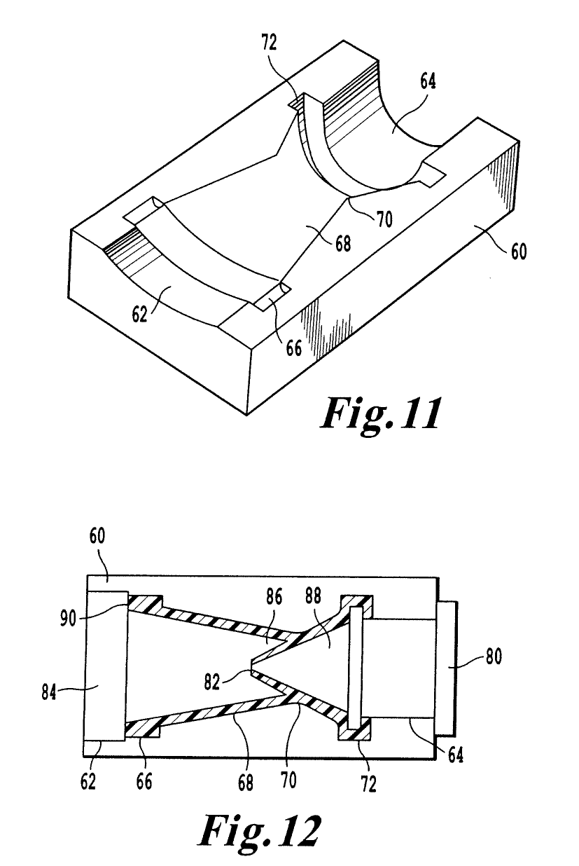

FIG. 11 depicts an exemplary mold 60 in which an integrated closing valve and seal assembly can be made. FIG. 12 depicts an integrated closing valve and seal assembly 90 formed within the mold 60. The mold 60 can include a first neck portion 62 for forming a first excess portion 84 of the closing valve. The mold 60 can also include a second neck portion 64 for forming a second excess portion 80 of the seal assembly. The first excess portion 84 and the second excess portion 80 can be removed when the integrated closing valve and seal assembly 90 are removed from the mold 60. Alternatively, the material forming the integrated closing valve and seal assembly 90 can be inserted into the mold 60 such that the first excess portion 84 and the second excess portion 80 are not formed.

The mold 60 includes a first rim portion 66 and a closing valve body portion 68 for forming the closing valve. The mold 60 also includes a joining portion 70 and a second rim portion 72 for forming an upper portion of the seal assembly. A closing valve insert 86 and a seal assembly insert 88 can be inserted into the mold 60 to help shape the material forming the integrated closing valve and seal assembly 90. A lower portion of the seal assembly 82 can be formed between the closing valve insert 86 and the seal assembly insert 88. Molding procedures known to a person of ordinary skill in the art can be used to form the various embodiments described herein. Additionally, the mold 60 shown in FIGS. 11 and 12 can be modified to form the various shapes of the embodiments described herein.

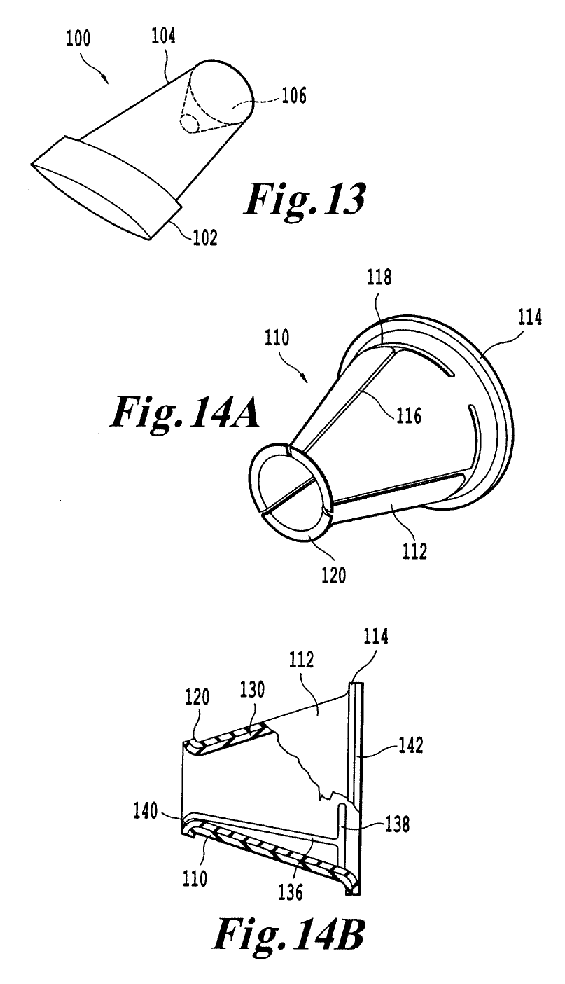

FIG. 13 depicts another exemplary closing valve 100. Compared with the closing valve 10 shown in FIG. 1, the closing valve 100 includes a thick outer lip 102, and the closing valve body 104 has a conical shape. The outer lip 102 of the closing valve 100 can be thicker than the closing valve body 104. Additionally, the thick outer lip 102 has a flat opening to assist in coupling the closing valve 100 to a capsule (not shown). FIG. 13 also depicts a portion of an exemplary seal assembly 106 inside the closing valve 100.

FIG. 14A depicts a first seal 112 of an exemplary seal assembly 110. The first seal 112 includes a plurality of lengthwise slots 116 terminating against circumferential slots 118. The first seal 112 also includes a rim portion 114 and a lip portion 120. The lip portion 120 of the first seal 112 has a bell shape such that the lip portion 120 turns outward from the body of the first seal 112.

FIG. 14B depicts the first seal 112 and a second seal 130 of the seal assembly 110. The first seal 112 and the second seal 130 can be arranged coaxially. The second seal 130 also includes a plurality of lengthwise slots 136 terminating against circumferential slots 138. The second seal 130 also includes a rim portion 142 and a lip portion 140. The lip portion 140 of the second seal 130 has a bell shape such that the lip portion 140 turns outward from the body of the second seal 130.

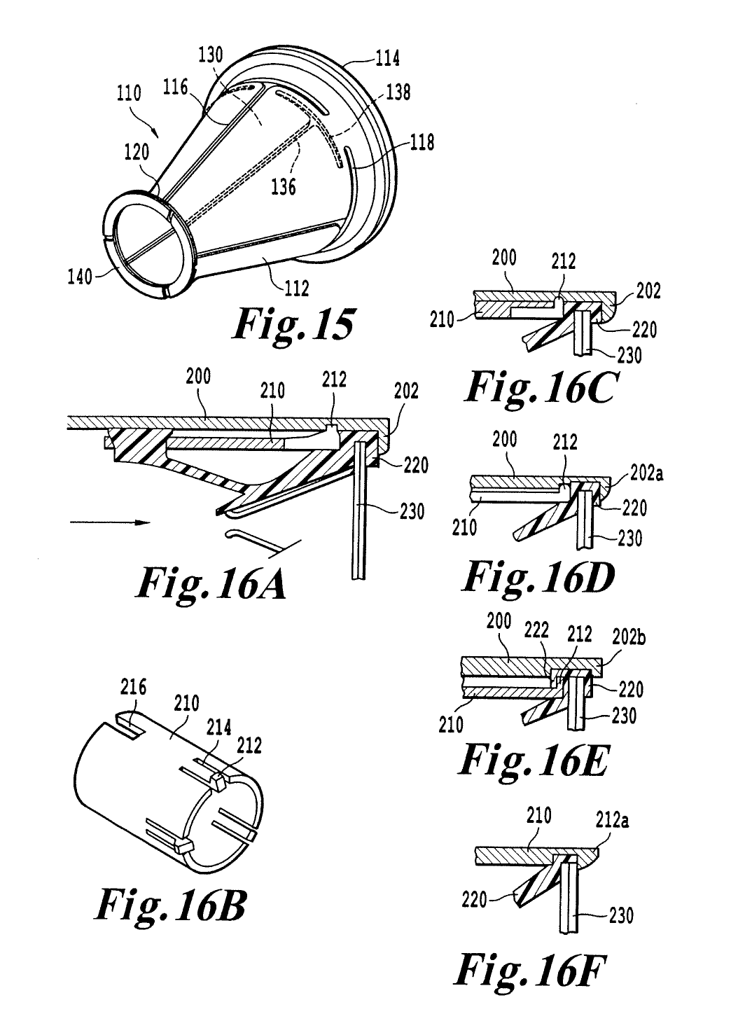

FIG. 15 depicts the first seal 112 and the second seal 130 of the seal assembly 110. The lengthwise slots 136 of the second seal 130 are offset with respect to the lengthwise slots 116 of the first seal 112 by a predetermined amount. The offset shown in FIG. 15 is approximately 30°. FIG. 16A depicts an exemplary integrated closing valve and seal assembly 220 with seals 230 coupled thereto. The seal assembly 220 is positioned within an exemplary capsule 210. The capsule 210 includes a coupling member 212. The seal assembly 220 and the capsule 210 are positioned within an exemplary cannula 200. The cannula 200 includes an end member 202. In the embodiment shown in FIG. 16A, the seal assembly 220 can be inserted into the cannula 200 such that the seal assembly 220 is positioned against the end member 202 of the cannula 200. The coupling member 212 of the capsule 210 fits against a receiving area of the cannula 200, thereby coupling the cannula 200, the capsule 210, and the seal assembly 220. Such a receiving area of the cannula 200 can be a recessed portion or another suitable portion to removably hold the coupling member 212.

FIG. 16B depicts the capsule 210 shown in FIG. 16A. The capsule 210 includes a plurality of coupling members 212 and coupling slots 214 bordering the coupling members 212. The coupling slots 214 allow the coupling members 212 to be flexible enough to fit within the cannula 200, as shown in FIG. 16A. The capsule 210 also includes at least one slot 216 to hold a lip portion of the closing valve (not shown).

FIG. 16C depicts another exemplary embodiment of a seal assembly 220 and capsule 210 integrated with a cannula 200. In this embodiment, an end member 202 of the cannula 200 extends radially inward a same distance as an end portion of the seal assembly 220.

FIG. 16D illustrates yet another exemplary embodiment of a seal assembly 220 and capsule 210 integrated with a cannula 200. In this embodiment, an end member 202a of the cannula 200 does not extend as far radially inward as the end member 202 shown in FIG. 16C.

FIG. 16E illustrates another exemplary embodiment of a seal assembly 220 and capsule 210 integrated with a cannula 200. In this embodiment, the coupling member 212 fits within a coupling portion 222 of the seal assembly 220. Further, the coupling portion 222 of the seal assembly 220 fits into a receiving area of the cannula 200, thereby coupling the cannula 200, the capsule 210, and the seal assembly 220. Additionally, an end member 202b of the cannula 200 does not extend as far radially inward as the end member 202 or the end member 202a.

FIG. 16F illustrates an exemplary embodiment of an integrated seal assembly 220 and a capsule 210. The capsule 210 includes a coupling member 212a that interacts with the seal assembly 220. Thus, the seal assembly 220 can be coupled to the capsule 210 such that the closing valve (not show) does not have to be fixed to the capsule 210. In another exemplary embodiment, the coupling member 212a could extend further radially inward such that the coupling member holds the seals 230 against the seal assembly 220.

As can thus be appreciated, the above-noted structure serves to provide an integrated valve assembly which can be insertable within a trocar and can be sold either assembled with a trocar or sold separately therefrom, particularly with respect to reusable trocars of the type typically marketed in Europe. Such can also be modified so as to replace the existing seals within presently available trocars so as to improve their performance.

Obviously, numerous modifications and variations of the present invention are possible in light of the above teachings. It is therefore to be understood that within the scope of the appended claims, the invention may be practiced otherwise than as specifically described herein.

What is claimed is:

1. A seal assembly, comprising:
a first seal including at least one longitudinal slot therein;
a second seal including at least one longitudinal slot therein, positioned coaxially with the first seal; and
a positioning element positioned coaxially with the first seal,
wherein the at least one longitudinal slot of the first seal is offset from the at least one longitudinal slot of the second seal in a radial direction from a longitudinal axis of the seal assembly.

2. The seal assembly according to claim 1, further comprising:
a closing valve disposed to the seal assembly.

3. The seal assembly according to claim 1, wherein an inner rim of the first seal has a substantially cylindrical shape and is configured to engage the positioning element during passage of a surgical tool along the longitudinal axis of the seal assembly.

4. The seal assembly according to claim 1, wherein the second seal extends radially a shorter distance than the first seal.

5. The seal assembly according to claim 1, wherein
the first seal includes at least one circumferential slot extending in a transverse direction with respect to the at least one longitudinal slot of the first seal, and
the second seal includes at least one circumferential slot extending in a transverse direction with respect to the at least one longitudinal slot of the second seal.

6. The seal assembly according to claim 1, wherein the at least one longitudinal slot of the first seal is offset from the at least one longitudinal slot of the second seal by 60 degrees in a radial direction from a longitudinal axis of the seal assembly.

7. The seal assembly according to claim 1, wherein the at least one longitudinal slot of the first seal is offset from the at least one longitudinal slot of the second seal by 30 degrees in a radial direction from a longitudinal axis of the seal assembly.

8. The seal assembly according to claim 1, wherein
the second seal includes three lengthwise slots therein and the three lengthwise slots are offset from one another by 120 degrees,
the first seal includes three lengthwise slots therein and the three lengthwise slots are offset from one another by 120 degrees, and
each of the three lengthwise slots of the first seal is offset from two of the lengthwise slots of the second seal by 60 degrees.

9. The seal assembly according to claim 1, wherein the seal assembly is attached to a capsule.

10. The seal assembly according to claim 1, wherein
the first seal has a substantially conical shape, and
the second seal has a substantially conical shape.

11. The seal assembly according to claim 1, wherein
a lip portion of the first seal has a substantially bell shape, and
a lip portion the second seal has a substantially bell shape.

12. An integrated assembly, comprising:
a first seal including at least one longitudinal slot therein;
a second seal including at least one longitudinal slot therein, positioned coaxially with the first seal;
a positioning element positioned coaxially with the first seal; and
a closing valve disposed to the positioning element and configured to close an opening of the integrated assembly,
wherein the at least one longitudinal slot of the first seal is offset from the at least one longitudinal slot of the second seal in a radial direction from a longitudinal axis of the integrated assembly.

13. The integrated assembly according to claim 12, wherein an inner rim of the first seal has a substantially cylindrical shape and is configured to engage the positioning element during passage of a surgical tool along the longitudinal axis of the seal assembly.

14. The integrated assembly according to claim 12, wherein the second seal extends radially a shorter distance than the first seal.

15. The integrated assembly according to claim 12, wherein
the first seal includes at least one circumferential slot extending in a transverse direction with respect to the at least one longitudinal slot of the first seal, and
the second seal includes at least one circumferential slot extending in a transverse direction with respect to the at least one longitudinal slot of the second seal.

16. The integrated assembly according to claim 12, wherein the at least one longitudinal slot of the first seal is offset from the at least one longitudinal slot of the second seal by 60 degrees in a radial direction from a longitudinal axis of the integrated assembly.

17. The integrated assembly according to claim 12, wherein the closing valve includes at least one groove on an inside edge of the closing valve.

18. The integrated assembly according to claim 17, wherein the closing valve is configured to be stretched via the at least one groove.

19. The integrated assembly according to claim 18, wherein, when the closing valve is stretched, a top and a bottom of the closing valve are in contact.

20. The integrated assembly according to claim 19, wherein the top and the bottom of the closing valve each include a thick portion at a center of the closing valve.

21. The integrated assembly according to claim 12, wherein the integrated assembly is attached to a capsule.

22. The integrated assembly according to claim 12, wherein
the first seal has a substantially conical shape, and
the second seal has a substantially conical shape.

23. The integrated assembly according to claim 12, wherein
the closing valve includes a closing valve body and a closing valve lip disposed on an end of the closing valve body, and
the closing valve lip is thicker than the closing valve body.

24. The integrated assembly according to claim 12, wherein
the closing valve is substantially flat.

25. An integrated assembly, comprising:
a first seal including at least one longitudinal slot therein;
a second seal including at least one longitudinal slot therein, positioned coaxially with the first seal;
a positioning element positioned coaxially with the first seal;
a closing valve disposed to the positioning element and configured to close an opening of the integrated assembly; and
a capsule attached to the closing valve,
wherein the at least one longitudinal slot of the first seal is offset from the at least one longitudinal slot of the second seal in a radial direction from a longitudinal axis of the integrated assembly.

26. The integrated assembly according to claim 25, wherein
the capsule includes two slots in an end portion thereof, and
the closing valve is attached to the two slots such that, in a first position, an opening of the closing valve is closed.

27. The integrated assembly according to claim 25, wherein the closing valve is positioned within the capsule.

28. The integrated assembly according to claim 25, wherein, when a surgical tool enters the assembly, the second seal and the first seal are configured to open the positioning element and only an outer rim of the positioning element contacts the surgical tool.

29. The integrated assembly according to claim 26, wherein, when a surgical tool extends through the assembly, the closing valve and the positioning element are configured to provide a seal around the surgical tool and to prevent a fluid from passing through the integrated assembly.

30. The integrated assembly according to claim 25, wherein
the first seal has a substantially conical shape, and
the second seal has a substantially conical shape.

31. The integrated assembly according to claim 25, wherein
the capsule includes at least one coupling member configured to couple the integrated assembly to a cannula.

32. The integrated assembly according to claim 31, wherein
the coupling member is a flexible tab configured to be received within a recessed portion of the cannula.

33. The integrated assembly according to claim 25, wherein
the capsule includes at least one coupling member,
the positioning element includes a coupling portion,
the at least one coupling member is configured to fit within the coupling portion, and
the coupling member is configured to couple the integrated assembly to a cannula.

34. The integrated assembly according to claim 25, wherein
the capsule includes at least one coupling member configured to couple the positioning element to the capsule.

35. The integrated assembly according to claim 25, wherein
the capsule includes at least one coupling member configured to couple the first seal, the second seal, and the positioning element to the capsule.

36. A method of forming a seal assembly, comprising;
providing a first seal including at least one longitudinal slot therein;
providing a second seal, including at least one longitudinal slot therein, coaxially with the first seal;
providing a positioning element coaxially with the first seal; and
positioning the first seal and the second seal such that the at least one longitudinal slot on the second seal is offset with respect to the at least one longitudinal slot on the first seal.

37. The method of forming a seal assembly according to claim 36, wherein
the providing the second seal includes providing the second seal with at least one circumferential slot extending in a traverse direction with respect to the at least one longitudinal slot therein, and
the providing the first seal includes providing the first seal with at least one circumferential slot extending in a traverse direction with respect to the at least one longitudinal slot therein.

38. The method of forming a seal assembly according to claim 36, wherein
the positioning the second seal includes positioning the at least one slot on the second seal offset by sixty degrees to the at least one slot on the first seal.

39. The method of forming a seal assembly according to claim 36, wherein
the providing the first seal includes providing the first seal with a substantially conical shape, and
the providing the second seal includes providing the second seal with a substantially conical shape.

40. The method of forming a seal assembly according to claim 36, further comprising:
integrating a capsule with the closing valve.

41. A method of forming a seal assembly, comprising;
providing a first seal including three longitudinal slots therein offset from one another by 120 degrees;
providing a second seal, including three longitudinal slots therein offset from one another by 120 degrees, coaxially with the first seal;
providing a positioning element coaxially with the first seal; and
positioning the first seal and the second seal such each of the three longitudinal slots on the second seal are offset with respect to two of the longitudinal slots on the first seal by 60 degrees.

42. The method of forming a seal assembly according to claim 41, wherein
the providing the first seal includes providing the first seal with a substantially conical shape, and
the providing the second seal includes providing the second seal with a substantially conical shape.

43. The method of forming a seal assembly according to claim 41, further comprising:
integrating a capsule with the closing valve.

44. A method of forming an integrated assembly, comprising:
providing a first seal including at least one longitudinal slot therein;
providing a second seal, including at least one longitudinal slot therein, coaxially with the first seal;
providing a positioning element coaxially with the first seal;
integrating a closing valve distally to the positioning element; and
positioning the first seal and the second seal such that the at least one longitudinal slot on the second seal is offset with respect to the at least one longitudinal slot on the first seal.

45. The method of forming an integrated assembly according to claim 44, wherein
the providing the first seal includes providing the first seal with a substantially conical shape, and
the providing the second seal includes providing the second seal with a substantially conical shape.

46. The method of forming an integrated assembly according to claim 44, further comprising:
integrating a capsule with the closing valve.

47. The method of forming an integrated assembly according to claim 46, wherein the integrating a capsule includes positioning the closing valve within the capsule, stretching the closing valve such that a portion of the closing valve extends outside the capsule, fixing the closing valve to the capsule, and removing the portion of the closing valve that extends outside the capsule.

48. The method of forming an integrated assembly according to claim 44, further comprising:
forming the positioning element in a mold; and
forming the closing valve in the mold.
